(19) United States
(12) Patent Application Publication
(10) Pub. No.: US 2021/0022904 A1
(43) Pub. Date: Jan. 28, 2021

(54) NOVEL SYSTEM TO PREVENT INAPPROPRIATE MEDICAL TUBING REMOVAL

(71) Applicants: Mark Bayard Lyon, Chicago, IL (US); John Lyon Havlik, Mequon, WI (US)

(72) Inventors: Mark Bayard Lyon, Chicago, IL (US); John Lyon Havlik, Mequon, WI (US)

(21) Appl. No.: 16/520,353

(22) Filed: Jul. 24, 2019

(51) Int. Cl. A61F 5/37

(52) U.S. Cl. CPC A61F 5/37

(57) ABSTRACT

The present invention comprises a novel system to prevent inappropriate medical tubing removal by patients. The invention can be composed of a piece of clear plastic or thick fabric which can be held in place on the wearer by multiple belts. The device is designed to prevent the wearer from removing the medical tubing. When the device is worn and secured to the wearer, the wearer's hands are unable to intrude beneath the fabric to grasp the medical tubing and facilitate its removal.

### FIELD OF THE INVENTION

The present invention relates to a novel system to prevent inappropriate medical tubing removal. More particularly, the invention embodies an external wearable device that prevents easy access to medical tubing in the abdominal and pelvic regions by the wearer, preventing damage caused to the body from inappropriate removal of this tubing.

### BACKGROUND

Various types of medical tubing such as gastrostomy tubes and urinary catheters are commonly used in patients. Gastrostomy tubes are tubes that are inserted directly into the stomach which provide direct nutrition to patients that have difficulty obtaining this nutrition directly through eating. Catheters are used for a variety of reasons, including as channels to help patients urinate when a large prostate occludes the normal passage of urine.

An all-too-common problem with medical tubing is inappropriate removal. Patients in altered states of consciousness, in delirium, or with dementia will feel the tubing causing them irritation, pain, or discomfort and will often attempt to tug the tubing out of their bodies. This can lead to medical complications such as infection, serious injury, and death. Inadvertent removal is also possible by tripping over the medical tubing or getting the tubing caught on other objects such as door handles.

Current guidelines for keeping medical tubing intact in patients with mental conditions include constant supervision by medical personnel and the placement of multiple “decoy” tubes around the site of the implanted tube. The use of constant supervision and multiple excess tubes is expensive and often fails to prevent inappropriate tubing removal. As such, in many cases, healthcare providers will resort to external physical restraint placement on a patient's arms or legs. This physical restraint is detrimental to the health of the patient and often results in legal action against the hospital or healthcare provider.

The deficiencies of today's medical tubing protection techniques described above are intended to provide an overview of some of the problems of today's deficiencies in medical tubing protection, and are not intended to be exhaustive. Other problems with the state of the art and corresponding benefits of some of the various non-limiting embodiments may become further apparent upon review of the following detailed description.

### BRIEF SUMMARY OF THE INVENTION

A simplified summary is provided herein to help enable a basic or general understanding of various aspects of exemplary, non-limiting embodiments that follow in the more detailed description and the accompanying drawings. This summary is not intended, however, as an extensive or exhaustive overview. Instead, the sole purpose of this summary is to present some concepts related to some exemplary non-limiting embodiments in a simplified form as a prelude to the more detailed description of the various embodiments that follow.

A wearable device is provided that can be affixed to the front and sides of the human body to prevent inappropriate medical tubing removal. The device can include a sheet with an attachment device on each end, one towards the proximal end of the wearer and one towards the distal end of the wearer. The sheet can be made of vinyl sheeting or fabric, and can have perforations to allow for air flow to prevent excess discomfort. The sheet may be clear to allow examination of the patient, to allow inspection of the sites of tubing insertion, and to verify the tubing is still inserted. The sheet can be long enough and cover as much of the body as is necessary to prevent tubing removal by passage of the arms under the sheet to grab the tubing. The sheet can be made of a thick vinyl to prevent tearing and to provide sufficient heft to prevent folding.

These and other embodiments are described in more detail below.

### BRIEF DESCRIPTION OF THE DRAWINGS

FIG. 1 is a schematic perspective view of a wearer wearing the tubing removal prevention device around the torso and abdomen according to an embodiment designed to prevent gastrostomy tube removal. This embodiment can consist of a thick sheet or vinyl or fabric which can be perforated for breathability, with belts attached to the top and bottom; these belts can be buckled onto the wearer.

FIG. 2 is a schematic cross-sectional side view of the same tubing removal prevention device detailed in FIG. 1 on the same wearer. In this embodiment the fabric does not extend around the entire body, though in other embodiments this extension around the entire body may occur.

FIG. 3 is a schematic perspective view of a wearer wearing the tubing removal prevention device around the pelvis and legs according to an embodiment designed to prevent urinary catheter removal. This embodiment can consist of a thick sheet or vinyl or fabric which can be perforated for breathability, with belts attached to the top and bottom; these belts can be buckled or otherwise secured onto the wearer at proximal and distal securing points. There may be multiple other belts attached, e.g. one belt attached to each leg, in other embodiments.

FIG. 4 is a schematic cross-sectional side view of the same tubing removal prevention device detailed in FIG. 3 on the same wearer. In this embodiment the fabric does not extend around the entire body, though in other embodiments this extension around the entire body may occur.

### DETAILED DESCRIPTION OF THE INVENTION

The claimed subject matter is now described with reference to the drawings, wherein like reference numerals are used to refer to like elements throughout. In the following description, for purposes of explanation, numerous specific details are set forth in order to provide a thorough understanding of the claimed subject matter. It may be evident, however, that the claimed subject matter may be practiced without these specific details. In other instances, well-known structures and devices are shown in block diagram form in order to facilitate describing the claimed subject matter.

As used in this application, the terms “catheter,” “gastrostomy tube,” “urinary catheter,” or the like can refer to a flexible tube inserted into the human body to introduce or withdraw fluids.

Moreover, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion. As used in this application, the term “or” is intended to mean an inclusive “or” rather than an exclusive “or.” Therefore, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

As discussed in the background, among other things, current catheters and gastrostomy tubes tend to cause bodily injury when removed at inappropriate times by patients in altered mental states, and tend to cause tissue trauma and retained foreign particles (e.g., catheter balloon fragments) when patients in an altered mental state remove the catheter from the human body.

Current state-of-the-art treatments for preventing this inappropriate medical tubing removal include placing decoy tubing on the body and forcibly restraining patients. Decoy tubing is often ineffective in preventing inappropriate tubing extraction and wastes medical tubing. Patient restraints can lead to functional decline of patients, serious injury from falls, death from strangulation, and depression. Restraints can also lead to rupturing of blood vessels and problems with blood circulation. Hospitals can lose accreditation over excessive use of restraints.

At least partly in consideration of these deficiencies of conventional methods of preventing inappropriate medical tubing removal, various embodiments of a device to prevent inappropriate tubing removal are provided that provide a way to prevent this self-harm from inappropriate tubing removal while avoiding the issues outlined above with patient restraint. As noted herein, various strap or buckle devices can be incorporated into the device to secure the device to the patient.

Details of various other exemplary, non-limiting embodiments are provided below.

The present invention describes an apparatus and method for preventing removal of abdominal or pelvic medical tubing by the wearer. In situations of both abdominal and medical tubing placement, the apparatus is worn over the tubing and can prevent its removal by pulling, pushing, hitting, or other methods.

According to an exemplary embodiment, the apparatus includes a thick sheet of material placed over the patient's abdominal region. The material extends to cover the sides of the body to prevent the wearer's hands from reaching underneath the material and compromising the removal prevention effect of the material. This material is held in place by two belts, one on the top of the material and one on the bottom of the material. When tension is applied to the belts and they are secured snugly around the wearer, the wearer is unable to reach underneath the thick material to pull out the medical tubing. In an altered mental state, the wearer is unable to undo the belts and remove the wearable device.

According to another exemplary embodiment, the apparatus includes a thick sheet of material placed over the patient's pelvic region and the thighs. The material extends to cover the sides of the body to prevent the wearer's hands from reaching underneath the material and compromising the removal prevention effect of the material. This material is held in place by two belts, one on the top of the material and one on the bottom of the material. When tension is applied to the belts and they are secured snugly around the wearer, the wearer is unable to reach underneath the thick material to pull out the medical tubing. In an altered mental state, the wearer is unable to undo the belts and remove the wearable device.

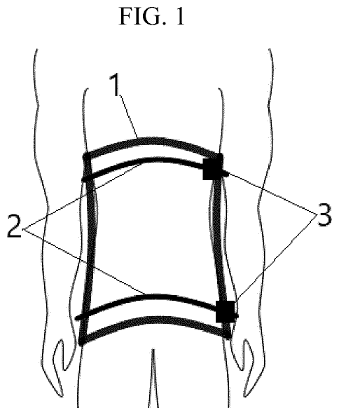

Referring now to the drawings, with reference initially to FIG. 1, a thick sheet of transparent fabric which is outlined with 1 according to an exemplary embodiment can include perforations for ventilation and two belts 2 one near the top of the fabric and one near the bottom of the fabric. The fabric 1 can be made of a clear vinyl or similar fabric. The fabric 1 can be perforated to prevent humidity buildup and allow a clear view of the affected area. The fabric can extend from the distal end of the pectoralis muscle to the proximal side of the pelvic region, and can cover the entirety of the abdominal region. The belts can include a buckle 3 of which many common varieties are suitable for quick and easy securing to the body.

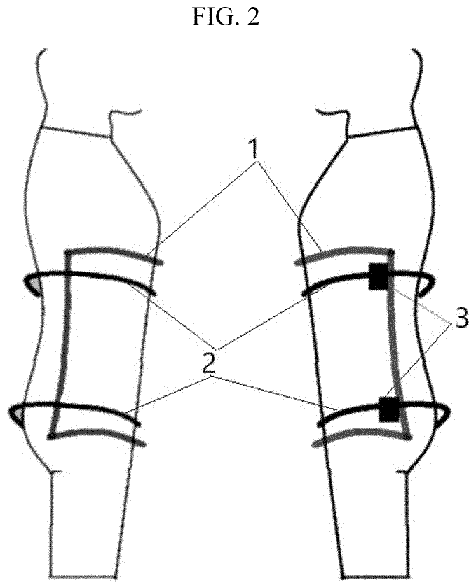

According to this exemplary embodiment, as shown in FIG. 2, the fabric which is outlined in 4 can extend around the body covering almost the entirety of the sides of the abdominal region. This will prevent hand insertion underneath the fabric 4. The belts 5 are buckled on the side of the body by buckles 6. These belts secure the fabric to the body and prevent hand intrusion under the fabric 4.

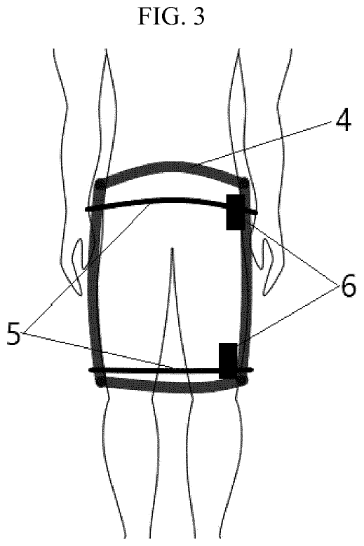

According to another exemplary embodiment, with reference to FIG. 3, a thick sheet of transparent fabric which is outlined in 1 according to an exemplary embodiment can include perforations for ventilation and two belts 2 one near the top of the fabric and one near the bottom of the fabric. The fabric 1 can be made of a clear vinyl or similar fabric. The fabric 1 can be perforated to prevent humidity buildup and allow a clear view of the medical tubing underneath. The fabric can extend from the middle of the abdominal region to the proximal side of the knee and can cover the entirety of the pelvic region. The belts can include a buckle 3 of which many common varieties are suitable for quick and easy securing to the body.

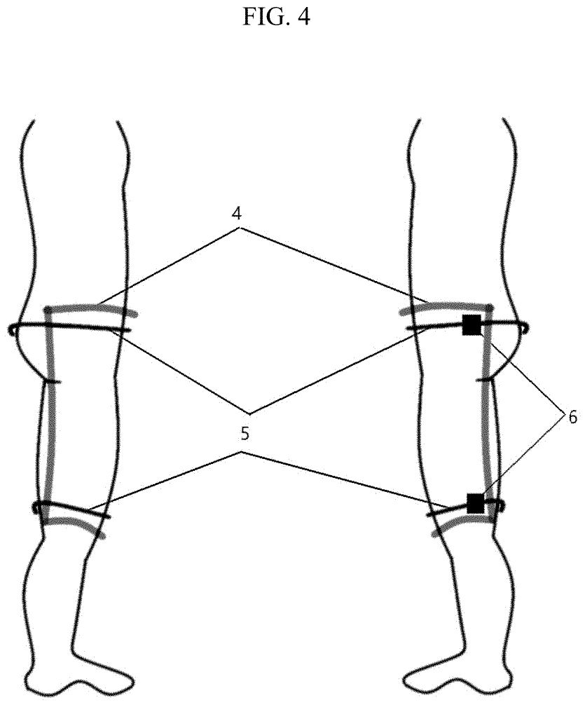

According to this exemplary embodiment, as shown in FIG. 4, the fabric which is outlined in 4 can extend around the body covering almost the entirety of the sides of the pelvic region. This will prevent hand insertion underneath the fabric 4. The belts 5 are buckled on the side of the body by buckles 6. These belts secure the fabric to the body and prevent hand intrusion under the fabric 4.

The word “exemplary” is used herein to mean serving as an example, instance, or illustration. For the avoidance of doubt, the subject matter disclosed herein is not limited by such examples. In addition, any aspect or design described herein as “exemplary” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, for the avoidance of doubt, such terms are intended to be inclusive in a manner similar to the term “comprising” as an open transition word without precluding any additional or other elements.

While the various embodiments have been described in connection with the preferred embodiments of the various figures, it is to be understood that other similar embodiments may be used or modifications and additions may be made to the described embodiment for performing the same function without deviating therefrom. Therefore, the present invention should not be limited to any single embodiment, but rather should be construed in breadth and scope in accordance with the appended claims.

What is claimed is:

1. A wearable device consisting of a thick piece of fabric or flexible plastic attached to the wearer by belts, straps, or similar devices which prevents inappropriate medical tubing removal from the abdominal or pelvic regions of the body by the wearer.
